(19) United States
(12) Patent Application Publication
(10) Pub. No.: US 2024/0361319 A1
(43) Pub. Date: Oct. 31, 2024

(54) IMMUNOFOCUS ASSAY FOR DETERMINING THE TITER OF DENGUE VIRUSES

(71) Applicant: TAKEDA VACCINES, INC., Cambridge, MA (US)

(72) Inventors: Betty Elena LUY, Cambridge, MA (US); Shi Hsia HWA, Cambridge, MA (US); Steven Michael ERB, Cambridge, MA (US); Jill A. LIVENGOOD, Cambridge, MA (US); Andrew Montgomery THOMSON, Singapore (SG)

(21) Appl. No.: 18/687,763

(22) PCT Filed: Sep. 9, 2022
(86) PCT No.: PCT/US2022/076151

§ 371 Date: Feb. 28, 2024

(30) Foreign Application Priority Data
Sep. 10, 2021 (EP) 21196157.8

(51) Int. Cl. G01N 33/569

(52) U.S. Cl. CPC G01N 33/56983; G01N 2333/185; G01N 2469/10

(57) ABSTRACT

The present invention provides improved immunofocus assays for determining the virus titer of dengue viruses and the use thereof in the quality control of vaccines based on live attenuated dengue viruses or in the diagnosis of a blood sample from a dengue infected individual.

### CROSS REFERENCE TO RELATED APPLICATIONS

This application is an International Patent Cooperation Treaty (PCT) application claiming priority to, and the benefit of European Patent Application No. 21196157.8, filed Sep. 10, 2021, the entire contents of which are incorporated herein by reference in its entirety.

### SEQUENCE LISTING

This application incorporates by reference in its entirety the Sequence Listing entitled “T08353WO—Sequence Listing—Final.XML”, which was created on Sep. 7, 2022, and filed electronically herewith.

### TECHNICAL FIELD

The present invention relates to improved immunofocus assays for determining titer of dengue viruses and the use thereof in the quality control of dengue vaccines, such as vaccines based on live attenuated dengue viruses. The assay may be used also for the determination of the titer of wild-type viruses.

### BACKGROUND OF THE INVENTION

Vaccines for protection against viral infections have been effectively used to reduce the incidence of human disease. One of the most successful technologies for viral vaccines is to immunize animals or humans with a weakened or attenuated virus strain (a “live attenuated virus”). The limited viral replication is sufficient to express the full repertoire of viral antigens and can generate potent and long-lasting immune responses to the virus. Thus, upon subsequent exposure to a pathogenic virus strain, the immunized individual is protected from the disease. These live attenuated viral vaccines are among the most successful vaccines used in public health.

Dengue disease is a mosquito-borne disease caused by infection with a dengue virus. Dengue virus infections can lead to debilitating and painful symptoms, including a sudden high fever, headaches, joint and muscle pain, nausea, vomiting and skin rashes. To date, four serotypes of dengue virus have been identified: dengue-1 (DENV-1), dengue-2 (DENV-2), dengue-3 (DENV-3) and dengue-4 (DENV-4). Dengue virus serotypes 1-4 can also cause dengue hemorrhagic fever (DHF) and dengue shock syndrome (DSS). In the most severe cases, DHF and DSS can be life threatening. Dengue viruses cause 50-100 million cases of debilitating dengue fever, 500,000 cases of DHF/DSS, and more than 20,000 deaths each year, a large portion of which are children. All four dengue virus serotypes are endemic throughout the tropical regions of the world and constitute the most significant mosquito-borne viral threat to humans there. Dengue viruses are transmitted to humans primarily by Aedes aegyptimosquitoes, but also by Aedes albopictus mosquitoes. Infection with one dengue virus serotype results in life-long protection from re-infection by that serotype, but does not prevent secondary infection by one of the other three dengue virus serotypes. In fact, previous infection with one dengue virus serotype may lead to an increased risk of severe disease (DHF/DSS) upon secondary infection with a different serotype.

Takeda has developed a tetravalent dengue vaccine (TAK-003/“QDENGA®”). The tetravalent dengue virus composition is a dengue virus composition comprising four different immunogenic components from the four different dengue serotypes DENV-1, DENV-2, DENV-3 and DENV-4, comprising four different live, attenuated dengue viruses, each representing one dengue serotype, and which aims to stimulate immune responses to all four dengue serotypes.

For quality control and reliable manufacture of vaccines including live attenuated viruses it is of utmost importance to determine the virus titer of the individual attenuated viruses, for example in the monovalent Bulk Drug Substance (BDS), and tetravalent vaccine drug product (DP). The determination of the virus titer can also be used as an in process control test (IPC) during manufacture.

For tetravalent virus compositions or any multivalent combination thereof, it is preferable to use the immunofocus assay (IFA) because the final formulated vaccine contains four different dengue virus serotypes and a method is required to identify foci generated by each serotype within the same sample.

The IFA has been introduced in the 1960s. The principle of the assay is based on classical vertebrate virus plaque assays where serial dilutions of virus are adsorbed on monolayers of adherent cells from a suitable host. After a period of time to allow infectious virions to bind and be taken up by cells, an overlay medium containing gelling agents is added to prevent diffusion of virions. Therefore, progeny virions can only infect cells adjacent to the original infected cell. This results in a roughly circular focus of infection for each infectious unit of virus.

The IFA differs from the classical plaque assay in that foci of infection are detected by immunostaining instead of visual observation of the cytopathic effect (CPE). After an incubation period to allow viral replication, cells are fixed and stained using virus-specific primary antibodies and a labeled secondary antibody.

In the prior art it was known to use e.g. high viscosity carboxymethyl cellulose (0.7%) [Brewoo et al., Vaccine 30 (2012), 1513-1520] or agarose (Lambeth et al., J. Clin. Microbiol. 43 (2005), 3267-3272) in the overlay medium of IFA for determining flaviviruses. However, the lack of sufficient resolution of the foci obtained by prior art methods can result in operator subjectivity in the recording of quantitative data, i.e. the focus counts.

Baer et al., J. Visualized Experiments no. 93 (2014), 1-10 describes that differing overlays and techniques can affect plaque formation and production. Plaque formation under different overlay media have been compared for three different virus species, i.e. Venezuelan Equine Encephalitis virus (VEEV), Influenza B Taiwan strain and Rift Valley fever virus (RVFV). The authors have used agarose and carboxymethyl cellulose (CMC) as solid or semi-solid overlay media or as a liquid overlay medium Avicel®. Only for VEEV viruses, agarose and liquid overlays performed superior compared to CMC. For RVFV, similar results have been obtained for agarose, CMC and Avicel® overlays have been obtained. For influenza B Taiwan strain, the use of an agarose overlay had the best performance. Consequently, in terms of clarity and sharpness of the plaques, the results significantly depend on the virus species used and cannot be predicted in advance. One technical problem underlying the present invention is to improve the qualitative appearance and reproducibility of the foci in order to reduce the operator subjectivity in the recording of quantitative data, i.e. focus counts. The improved quality of appearance of the foci and reproducibility are relevant, in particular when automated focus counting is desired.

### SUMMARY OF THE INVENTION

The technical problems underlying the invention are solved by the provision of the subject-matter as defined in the claims.

According to a first aspect, the present invention provides a method for determining the titer of a dengue virus serotype in a dengue virus-containing sample, the method comprising the steps of:
(a) seeding cells from a dengue-susceptible cell line in an assay plate and culturing the cells for a culture period:
(b) preparing serial dilutions of the dengue virus-containing sample:
(c) adding the serially diluted samples to the cells seeded and cultured in step (a) and incubating the cells over a first incubation period:
(d) providing an overlay medium for the cells incubated in step (c), wherein the overlay medium comprises (i) microcrystalline cellulose and (ii) carboxymethyl cellulose or a salt thereof, and incubating the cells with the overlay medium over a second incubation period;
(e) fixing of the incubated cells:
(f1) immunostaining of the incubated cells using a dengue virus serotype specific antibody as first antibody and a second antibody being specific for the first antibody and conjugated to an enzyme capable of converting a substrate to a visible dye or conjugated to a detectable label: or
(f2) immunostaining of the incubated cells using a dengue virus serotype specific antibody as first antibody conjugated to a detectable label; and
(g) determining the titer of each dengue virus serotype by counting the number of foci in each well of the assay plate.

In a second aspect the present invention provides the use of the method according to the invention in the quality control of a virus preparation or a vaccine composition.

In a third aspect the present invention provides the use of the method according to the invention in the diagnosis of a blood sample from a dengue infected individual.

The inventors surprisingly found that an overlay medium comprising microcrystalline cellulose (MCC) and carboxymethyl cellulose (CMC) increases the visual appearance of the foci compared to the use of CMC alone. Distinct circular foci were observed. The appearance of the foci could be further improved by using an overlay medium comprising MCC, low viscosity CMC and medium viscosity CMC. Thereby, the appearance of the foci was improved and the number of satellites or difficult to read foci was reduced. Further, flocculation of the overlay medium was prevented. The above advantages are important for visual and automated counting of the foci in an IFA. The advantages result in increased precision and reproducibility of the IFA. The improved immunofocus assay (IFA) can successfully be used to determine the infectious potency of Takeda's Tetravalent Dengue Vaccine (Takeda's TDV, tradename QDENGA®, Takeda's Development Code: TAK-003) monovalent samples.

The inventors have also found that the size and the number of the foci can be increased by adding a non-ionic detergent such as F127 to the incubation medium in the adsorption step of the IFA assay.

### BRIEF DESCRIPTION OF THE DRAWINGS

FIG. 1 shows the Genetic structure of the four dengue strains contained in TDV. The solid red triangles indicate the three attenuating mutations present in the 5′NCR, NS1 and NS3 proteins. The TDV-1, TDV-3 and TDV-4 strains are chimeric viruses where the prM and E genes from dengue serotype 1, 3 and 4, respectively, are inserted into the TDV-2 backbone.

FIG. 2 shows TDV-1 foci under different overlay media and incubation times. CMC: carboxymethyl cellulose: DPI: days post infection

FIG. 3 shows Foci/satellites: comparisons between Avicel® RC-591 and Avicel® RC-591 and CMC with TDV-2 and TDV-4.

FIG. 4 shows the virus titer of TDV-4 virus as determined by immunofocus assay. F127 Ads: Poloxamer 407 (PluronicR F127) contained in the adsorption medium: No F127: no Poloxamer 407 (PluronicR F127) present in the overlay medium and the adsorption medium: F127 Ads/Overlay: Poloxamer 407 (Pluronic® F127) contained in the adsorption medium and the overlay medium: F127 Overlay: Poloxamer 407 (PluronicR F127) contained in the overlay medium. Further details are described in the examples.

FIGS. 5A and 5B show the foci of each serotype in monovalent and tetravalent positive controls. Visual assessment shows that no difference can be seen between the high and low CMC overlay media preparation.

### DETAILED DESCRIPTION OF THE INVENTION

Where the term “comprise” or “comprising” is used in the present description and claims, it does not exclude other elements or steps. For the purpose of the present invention, the term “consisting of” is considered to be an optional embodiment of the term “comprising”. If hereinafter a group is defined to comprise at least a certain number of embodiments, this is also to be understood to disclose a group which optionally consists only of these embodiments.

Where an indefinite or a definite article is used when referring to a singular noun e.g. “a” or “an”, “the”, this includes a plural form of that noun unless specifically stated. Vice versa, when the plural form of a noun is used it refers also to the singular form.

Furthermore, the terms first, second, third or (a), (b), (c) and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

In the context of the present invention any numerical value indicated is typically associated with an interval of accuracy that the person skilled in the art will understand to still ensure the technical effect of the feature in question. As used herein, the deviation from the indicated numerical value is in the range of ±10%, and preferably of ±5%. The aforementioned deviation from the indicated numerical interval of ±10%, and preferably of ±5% is also indicated by the terms “about” and “approximately” used herein with respect to a numerical value.

### Dengue Virus

“Dengue virus” herein includes any wild-type or dengue virus mutant. The mutant may naturally occur or obtained by genetic engineering of the wild-type virus. The genetic modifications include additions, deletions, insertions and/or substitutions. The modifications are not particularly limited.

The term “dengue virus” also includes chimeras with genetic information from at least one further virus. The chimera may contain genetic information from another flavivirus such as Yellow Fever virus, Japanese Encephalitis virus, Murray Valley encephalitis virus, Zika virus and West Nile virus. Preferably, the chimera includes at least the E gene from another dengue virus strain.

Most preferably, the term “dengue chimera” is a dengue/dengue chimera derived from two different dengue viruses. Multivalent compositions comprising three or tetravalent compositions comprising four dengue/dengue chimeras are particularly envisaged.

The dengue virus is a single stranded, positive sense RNA virus of the family flaviviridae. The taxonomy is outlined in Table 1. The family flaviviridae includes three genera, flavivirus, hepacivirus and pestivirus. The genus flavivirus contains highly pathogenic and potentially hemorrhagic fever viruses, such as yellow fever virus and dengue virus, encephalitic viruses, such as Japanese encephalitis virus, Murray Valley encephalitis virus and West Nile virus, and a number of less pathogenic viruses.

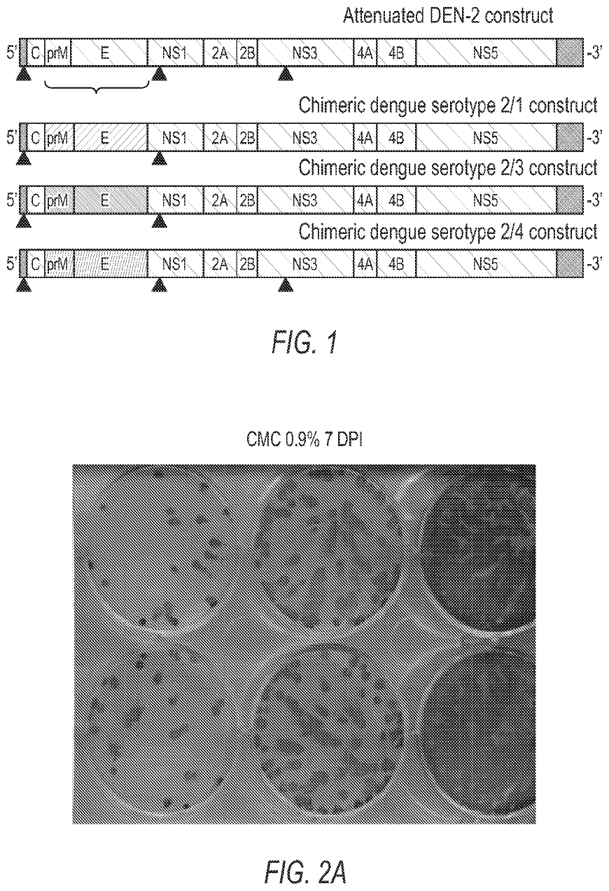

The flavivirus genome comprises in 5′ to 3′ direction (see FIG. 1):
a 5′-noncoding region (5′-NCR),
a capsid protein (C) encoding region,
a pre-membrane protein (prM) encoding region,
an envelope protein (E) encoding region,
a region encoding nonstructural proteins (NS1, NS2A, NS2B, NS3, NS4A, NS4B, NS5) and
a 3′ noncoding region (3′-NCR).

The viral structural proteins are C, prM and E, and the nonstructural proteins are NS1 to NS5. The structural and nonstructural proteins are translated as a single polyprotein and processed by cellular and viral proteases.

The dengue virus also includes dengue virus chimera comprising more than one dengue virus subtype. Suitable variants of dengue virus chimera are described in detail in WO 2020/051328, the disclosure of which is incorporated herein by reference. Preferably, the methods according to the invention are used for determining the virus titer of the dengue virus variants TDV-1, TDV-2, TDV-3 and TDV-4 (TAK-003/“QDENGA®”) outlined below.

The term also includes dengue virus chimera comprising genetic information from another flavivirus. Preferably, the other flavivirus is selected from Japanese encephalitis virus, Tick-borne encephalitis virus, West Nile virus, Yellow fever virus and Zika virus.

“Dengue virus vaccine composition” herein includes monovalent compositions comprising only a single dengue virus or a dengue virus chimera. It also includes a flavivirus chimera comprising at least the E gene from dengue virus. The term also includes multivalent composition comprising more than one single dengue virus or chimera or flavivirus chimera comprising at least the E gene from dengue virus. Preferably, the multivalent compositions include dengue virus from more than one subtype, wherein the subtype is selected from DENV-1, DENV-2, DENV-3 and DENV-4. More preferably, the multivalent composition is a tetravalent composition comprising dengue viruses and/or chimeras from each dengue virus subtype, such as e.g. a tetravalent composition comprising at least 3 live-attenuated dengue/dengue chimeras, such as e.g. 4 live-attenuated dengue-dengue chimeras or 3 live-attenuated dengue-dengue chimeras and one live-attenuated non-chimeric dengue strain (such as TAK-003/QDENGA®.

The dengue virus structural envelope (E) protein and pre-membrane (prM) protein have been identified as the primary antigens that elicit a neutralizing protective antibody response. For creation of the tetravalent dengue vaccine (TDV), TDV-2 was modified by replacing the nucleic acid sequence encoding the DENV-2 prM and E glycoproteins with the nucleic acid sequence encoding the corresponding wild type prM and E glycoproteins from the DENV-1, DENV-3, and DENV-4 wild type strains DENV-1 16007, DENV-3 16562 or DENV-4 1036 virus, respectively, using standard molecular genetic engineering methods (Huang et al. (2003) J. Virol. 77 (21): 11436-11447).

A diagram of the four TDV strains comprised in the dengue vaccine composition is shown in FIG. 1.

TABLE 2
Sequences characterizing the TDV virus strains

| SEQ ID NO. | dengue virus strain | sequence type |
|---|---|---|
| SEQ ID NO. 1 | TDV-1 | amino acid sequence |
| SEQ ID NO. 2 | TDV-2 | amino acid sequence |
| SEQ ID NO. 3 | TDV-3 | amino acid sequence |
| SEQ ID NO. 4 | TDV-4 | amino acid sequence |

### Methods for Determining the Titer of a Dengue Virus Serotype in a Dengue Virus-Containing Sample

According to the first aspect, the present invention provides a method for determining the titer of a dengue virus serotype in a dengue virus-containing sample, the method comprising the steps of:
(a) seeding cells from a dengue-susceptible cell line in an assay plate and culturing the cells for a culture period;
(b) preparing serial dilutions of the dengue virus-containing sample;
(c) adding the serially diluted samples to the cells seeded and cultured in step (a) and incubating the cells over a first incubation period;
(d) providing an overlay medium for the cells incubated in step (c), wherein the overlay medium comprises (i) microcrystalline cellulose and (ii) carboxymethyl cellulose or a salt thereof, and incubating the cells with the overlay medium over a second incubation period;
(e) fixing of the incubated cells;
(f1) immunostaining of the incubated cells using a dengue virus serotype specific antibody as first antibody and a second antibody being specific for the first antibody and conjugated to an enzyme capable of converting a substrate to a visible dye or conjugated to a detectable label; or
(f2) immunostaining of the incubated cells using a dengue virus serotype specific antibody as first antibody conjugated to a detectable label; and
(g) determining the titer of each dengue virus serotype by counting the number of foci in each well of the assay plate.

As used herein, a “dengue-susceptible cell line” is a cell line which can be kept in culture in vitro and which can be infected with dengue virus. In the context of the present invention the dengue-susceptible cell line is capable of being lysed upon replication of the virus in the cells so that plaques are formed. Suitable dengue-susceptible cell lines include, but are not limited to, Vero cells, LLC-MK2 cells and BHK-21 cells. Preferably, the dengue-susceptible cell line is a Vero cell line.

As used herein, “culturing” means maintaining cells under conditions selected such that the cells remain viable and able to divide. Such conditions include temperature, pH and culture medium. Typically, mammalian cells are cultured at a temperature of about 37° C.

The dengue-virus containing sample may be the virus containing samples as used in the culture of viruses for manufacturing purposes, as well as the bulk drug substance (BDS) containing the monovalent dengue virus serotype, mixtures of different serotypes and in particular the tetravalent final bulk drug product (BDP). The dengue-virus containing sample also includes serum samples obtained by collecting blood from a human subject and separating the serum from the other components of the blood.

In the method of the present invention serial dilutions of the dengue virus-containing sample are prepared. The serial dilution of the dengue virus-containing samples is the stepwise dilution of the sample according to a given dilution factor. In one embodiment, if the dengue virus-containing sample, is a monovalent BDS, the dilution factor may be between 1:20 to 1:125,000,000. In another embodiment, if the dengue virus-containing sample is the tetravalent dengue vaccine, the dilution factor may be between 1:20 to 1:62,500.

In one embodiment, the dengue-susceptible cell line used in step (a) is selected from Vero cells, LLC-MK2 cells and BHK-21 cells. Preferably, the dengue-susceptible cell line used in step (a) is a Vero cell line.

The dengue-susceptible cell line is seeded on 6 well plates or 12 well plates, i.e. a defined amount of the dengue-susceptible cell line is introduced into a well of a 96-well plate which contains a suitable growth medium for the dengue-susceptible cell line. Preferably, 6 well plates are used.

Suitable growth media for dengue-susceptible cell lines are known to the skilled person and include DMEM with 10% fetal bovine serum. The dengue-susceptible cell line is seeded with a density of 1 to 4×10⁵ cells per ml, preferably of 1.5 to 3.5×10⁵ cells per ml, more preferably of 2 to 3×10⁵ cells per ml and most preferably of 2.5×10⁵ cells per ml. Alternatively, it is preferred that the dengue-susceptible cell line is seeded in the plates with a density of 1 to 10×10⁴ cells/cm², preferably of 6 to 9×10⁴ cells/cm², more preferably of 8 to 9×10⁴ cells/cm² and most preferably of about 8.31×10⁴ cells/cm². In some embodiments, the dengue-susceptible cell line is cultured for a culture period of 24 to 72 hours, preferably 36 to 60 hours and most preferably of 40 to 48 hours. Alternatively, it is preferred that the culture period is from 18 to 30 hours more preferably, from 22 to 26 hours. The culture period is calculated from the time the cells are seeded until the time the separate mixtures of the serially diluted dengue virus-containing samples are added to the cells. Preferably, the cells are grown to at least 90%, more preferably at least 95% confluence.

The serially diluted dengue virus-containing samples prepared in step (c) are added to the dengue-susceptible cell line to allow for virus adsorption. The cells are incubated with the serially diluted samples for a period of 1 to 10 hours, more preferably 2 to 8 hours, even more preferably, 5.5 to 6.5 hours at a temperature of about 37° C.

In a preferred embodiment the first incubation in step (c) is carried out in a medium containing a non-ionic detergent, preferably the non-ionic detergent is a block copolymer of ethylene oxide and propylene oxide, more preferably the non-ionic detergent is F127 (also designated as Pluronic® F 127, poloxamer 407, Kolliphor® P 407 and Lutrol® F 127).

Optionally, the overlay medium in step (d) additionally contains a non-ionic detergent, preferably the non-ionic detergent is a block copolymer of ethylene oxide and propylene oxide, more preferably the non-ionic detergent is F127 (also designated as Pluronic F127, poloxamer 407, Kolliphor P407 and Lutrol F127).

In a more preferred embodiment the final concentration of the non-ionic detergent in the incubation medium is in the range from 0.01% (v/v) to 2% (v/v), preferably from 0.05% (v/v) to 0.15% (v/v), most preferably 0.1% (v/v). In another preferred embodiment the final concentration of the non-ionic detergent in the overlay medium is in the range from 0.01% (v/v) to 2% (v/v), preferably from 0.05% (v/v) to 0.15% (v/v), most preferably 0.1% (v/v).

The overlay provided in step (d) to the incubated cells serves to limit the virus diffusion within the plate which permits plaque formation. The overlay is added without aspiration directly to the virus dilutions. Generally, the overlay medium in step (d) comprises microcrystalline cellulose and a first carboxymethyl cellulose, or a salt thereof.

“Microcrystalline cellulose” (MCC) is a pure partially depolymerized cellulose synthesized from α-cellulose precursor (type Iβ), obtained as a pulp from fibrous plant material, with mineral acids using hydrochloric acid to reduce the degree of polymerization. The MCC can be synthesized by different processes such as reactive extrusion, enzyme mediated, steam explosion, and acid hydrolysis. It is commonly manufactured by spray-drying the neutralized aqueous slurry of hydrolyzed cellulose. Preferably, the MCC is of pharmaceutical Grade such as the commercially available as Avicel® PH 101, PH 102, PH 103, PH 105, PH 112, PH 113, PH 200 and PH 301 types of MCCs from FMC Biopolymer.

“Carboxymethyl cellulose” (CMC) is a cellulose derivative with carboxymethyl groups bound to some of the hydroxyl groups the cellulose. It is synthesized by the alkali-catalyzed reaction of cellulose with chloroacetic acid. The carboxymethyl cellulose may be in the form of a salt. Herein, any salts thereof are included, preferably the salt is a sodium, potassium, magnesium or calcium salt of carboxymethyl cellulose. Particularly preferred, the salt may be a sodium carboxymethyl cellulose salt.

In a preferred embodiment, the overlay medium is obtained by dissolving of Avicel® RC/CL. Avicel RC/CL is commercially available from FMC Biopolymer (DuPont). Avicel RC/CL is a colloidal, water-dispersible, spray-dried blend of MCC and CMC, wherein the CMC is a medium viscosity sodium carboxymethyl cellulose. It has a composition of 82% to 89% (w/w) MCC and 11% to 18% (w/w) medium viscosity CMC on a dry basis. More preferably, the Avicel RC/CL is RC-501, RC-581, RC-591 and CL-611 which differ in their NaCMC content particle size and viscosity. Most preferred, the Avicel RC/CL is Avicel RC-591. Alternatively, Neocel® from Mingtai Chemical, Co. Ltd., Tabulose® from Roquette Freres (Roquettes) may be used.

Preferably, the overlay medium comprises:
(a) 0.4% to 1.8% (w/v) microcrystalline cellulose; and
(b) 0.2% to 1.2% (w/v) carboxymethyl cellulose, or a salt thereof.

More preferably, the overlay medium comprises:
(a) 0.8% to 1% (w/v) microcrystalline cellulose; and
(b) 0.4 to 0.6% (w/v) carboxymethyl cellulose, or a salt thereof.

In a more preferred embodiment the overlay medium comprises two different types of CMC. More preferably, a medium viscosity CMC and a low viscosity CMC, or salts thereof: or a medium viscosity CMC and a high viscosity CMC, or salts thereof. Particularly preferred, the overlay medium comprises a medium viscosity CMC and a low viscosity CMC, or a salts thereof. Alternatively, it is preferred that the overlay medium comprises two different types of medium viscosity CMC. If the microcrystalline cellulose and the first medium viscosity CMC is provided in the form of Avicel® RC-591 (Dupont), the second medium viscosity CMC can be provided e.g. in the form of a medium viscosity (Sigma, C4888).

“High viscosity CMC” means that a 1% (w/v) CMC solution exhibits a viscosity of 5000 to 8500 Pa·S at 25° C.: a “medium viscosity CMC” exhibits as a 1% (w/v) solution a viscosity of 1000 to 5000 Pa·S at 25° C.; and a “low viscosity CMC” exhibits as a 1 % (w/v) solution a viscosity of 5 to 1000 Pa·S at 25° C. Preferably, the viscosity is determined using the Brookfield viscometer. Different types of CMC are commercially available.

In a preferred embodiment the overlay medium is prepared by dissolving Avicel® RC/CL, in particular Avicel® RC-591, such that a final Avicel concentration of 1% (w/v) is prepared. In a more preferred embodiment, in addition to the dissolution of Avicel® as described above, a low viscosity or high viscosity CMC is dissolved to prepare a final low or high viscosity CMC concentration of 0.35% (w/v) is prepared. Preferably, the second CMC is a low viscosity CMC, more preferably the second CMC is a sodium salt low viscosity CMC. Alternatively, preferably the second CMC is a high viscosity CMC such as available from Sigma, Cat. No. C5013.

In one embodiment, different second incubation periods are used in step (d) for the different dengue serotypes. Preferably, if a monovalent sample is tested, the incubation periods for dengue serotypes may be 20 to 130 hours. A low incubation period is sufficient if a fluorescent label is used.

More preferably, if a monovalent sample is tested, the incubation periods for dengue serotypes 1, 2 and 3 are 93±6 hours; and for dengue serotype 4 the incubation period is 117±6 hours. Further preferably, if a tetravalent sample containing each of dengue serotypes 1 to 4 is tested, the incubation periods for dengue serotypes 2 and 3 are 93±6 hours: and the incubation periods for dengue serotypes 1 and 4 are 117±6 hours. Alternatively, for all four serotypes an incubation period of 96±6 hours is used.

In step (e) the incubated cells of step (d) are fixed. Fixation is generally performed after mechanical removal of the overlay medium. This can be done by aspiration or decanting. Fixation of cells may be performed by methods known in the art. Preferably, the cells are fixed using methanol. Alternatively, acetone or paraformaldehyde may be used. After fixation the plates may be dried.

In step (f1) the incubated cells are immunostained using a dengue virus serotype specific antibody as first antibody and a second antibody being specific for the first antibody and conjugated to an enzyme capable of converting a substrate to a visible dye.

Alternatively, the second antibody may be conjugated to a detectable label.

In an alternative embodiment (f2), the dengue virus serotype specific antibody is directly conjugated to a detectable label. In this embodiment there is no need for a secondary labelled antibody.

The term “detectable label”, as used herein, refers to any compound or moiety that comprises one or more appropriate chemical substances or enzymes, which directly or indirectly generate a detectable compound or signal in a chemical, physical or enzymatic reaction. Labeling can be achieved by methods well known in the art (see, for example, Lottspeich, F., and Zorbas H., Springer Spektrum 2012, Bioanalytik).

According to one embodiment of the invention the detectable label is selected from the group consisting of fluorescent labels, magnetic labels, enzyme labels, colored labels, chromogenic labels, luminescent labels, radioactive labels, haptens, biotin, metal complexes, metals, and colloidal gold. All these types of labels are well established in the art.

Suitable fluorescent dyes include Alexa Fluor®, Dy Light®, FITC, TRITC, rhodamine, Oregon Green® and Texas Red®.

The skilled person knows how to prepare serotype-specific antibodies. Suitable approaches are described for example in Gentry et al. (1982) Am. J. Trop. Med. Hyg. 31, 548-555: Henchal et al. (1985) Am. J. Trop. Med. Hyg. 34, 162-169; and Henchal et al. (1982) Am. J. Trop. Med. Hyg. 31(4): 830-6). For example, mice can be immunized with a specific dengue serotype and the B cells isolated from these mice can be fused with a fusion partner to prepare a hybridoma. Suitable serotype-specific antibodies are selected based on the binding of the antibodies to the serotype with which the mice were immunized and lack of binding to those serotypes with which the mice were not immunized. In one embodiment, the mice were immunized with a serotype selected from dengue 1 strain Hawaii, Envelope, dengue 2 strain New Guinea C, Envelope, isotype 1, dengue 3 strain H87, Envelope, isotype 2A, and dengue 4 strain H241, Envelope, isotype 2A.

Preferably, the serotype specific anti-dengue monoclonal antibodies are added to the corresponding wells and incubated for 60 to 90 minutes at 36±2° C., or 8±4 hours at 2-8° C., before the cells are washed and incubated with a secondary antibody conjugated to an enzyme. The secondary antibody binds to the serotype specific anti-dengue monoclonal antibodies for 45 to 75 minutes at 36° C.

The secondary antibody conjugated to an enzyme comprises a variable region being specific for the Fc part of the dengue serotype specific first antibody. The enzyme may an alkaline phosphatase or a peroxidase. Preferably, the enzyme is an alkaline phosphatase. Suitable substrates for alkaline phosphatase which convert into a visible dye are known to the person skilled in the art. Preferably, the substrate is BCIP (5-Bromo-4-chloro-3-indolyl phosphate)/NBT (4-nitro blue tetrazolium chloride). If a peroxidase is used as the enzyme, the substrate may be 2-amino-9-ethyl carbazole (AEC).

If BCIP/NBT is used as the substrate, the solution is added at 0.5 ml/well and the plates are incubated at room temperature until dark blue or purple foci are clearly visible. The reaction is stopped by washing with distilled water and the plates are allowed to air dry at room temperature.

The number of foci may be determined visually or using a plaque counter such as the ViruSpot Plaque counter.

After all SST criteria are met, all wells are counted, and the foci count is documented within 0 to ≤100. Using automated focus counting, foci counts of more than 100 are envisaged. Preferably, the foci counts may be up to 200, more preferably up to 160 foci per plate. Two overlapping foci are counted as one foci if the distance between their center is smaller than the radius. Only wells with a foci count of 10 to 100 are used for data evaluation. Number rounding is avoided in the calculations and the result is rounded at the end of the calculation to 1 or 2 decimal places, in accordance with the test plan or specification. The final results are reported as the rounded mean value.

The viral titers are calculated as follows:
For Takeda's TOV Monovalent Sample (PFU/mL):

$$Titer = \frac{{\sum }_{Wells with 10 to 100 faci} ( Focus count \times Dilution factor )}{\begin{matrix}( Inoculum volume 0.5 mL / well ) \times \\ ( Number of wells with 10 - 100 faci )\end{matrix}}$$

For Takeda's TOV Tetravalent sample (PFU/0.5 ml dose):

$$Titer = \frac{{\sum }_{Wells with 10 to 100 faci} ( Focus count \times Dilution factor )}{\begin{matrix}( Inoculum volume 0.5 mL / well ) \times \\ ( Number of wells with 10 - 100 faci )\end{matrix}} \times 0.5 mL dose$$

It is contemplated that the methods of the invention are useful for the determination of the titer of flaviviruses other than dengue virus. The flavivirus other than dengue virus may be selected from Japanese encephalitis virus, Tick-borne encephalitis virus, West Nile virus, Yellow fever virus and Zika virus.

In a further aspect the present invention provides the use of the inventive method in the quality control of a virus preparation or a vaccine composition. The reliable determination of the dengue virus titer is highly relevant at different stages of the vaccine manufacturing. It is used for assessing the quality of the different viral stocks. In addition, it is important for assessing the titer of the monovalent drug substance and before and after the final formulation of the tetravalent drug product. Preferably, the vaccine composition comprises four live attenuated dengue strains, such as TAK-003 (QDENGA®).

In a further aspect the present invention provides the use of the inventive method in the diagnosis of a blood sample from a Dengue infected individual. The method can therefore be determined whether a superinfection by two different dengue serotypes is present in the individual.

### EXAMPLES

The following Examples are included to demonstrate certain aspects and embodiments of the invention as described in the claims. It should be appreciated by those of skill in the art, however, that the following description is illustrative only and should not be taken in any way as a restriction of the invention.

### Dengue Virus Strains Used in the Examples

The methods used to generate the chimeric dengue strains TDV-1, -3 and -4 were standard molecular cloning and DNA engineering methods and are described in Huang et al. (2003) J. Virology 77(21): 11436-11447.

The amino acid sequences of TDV-1 to TDV-4 used in the present example are shown in Table 2 above and the accompanying sequence listing.

### General Protocol for Immunofocus Assay

Vero cells at approximately 1.33×10⁵ cells/mL are plated in a volume of 3 mL/well in 6-well tissue culture plates and incubated at 36±2° C., 5% CO2 for 2 days. Five-fold serial dilutions of the samples in medium consisting of Dulbecco's Modified Eagle Medium (DMEM) with 2% Fetal Bovine Serum (FBS), 2% L-Glutamine and 1% Kolliphor are prepared, such that the serial dilution brackets the specification range for each virus in the monovalent DS and/or tetravalent DP. For monovalent samples, there are additional pre-dilution steps before the 5-fold serial dilution. Growth medium is removed from the tissue culture plates and 500 μL per well of diluted sample is applied, i.e. one dilution series is applied to each well of the plate. Following virus adsorption for 6±0.5 hours at 36±2° C., 5% CO₂, overlay medium containing DMEM with 2% FBS, 2% L-Glutamine, 1% (w/v) Avicel® RC-591 and 0.35% (w/v) carboxymethyl cellulose (CMC) sodium salt, low viscosity (Sigma-Aldrich, Cat. No. C5678), is applied in a volume of 3 mL/well. For monovalent test articles, plates are incubated at 36±2° C., 5% CO₂ for 93±6 hours for TDV-1, TDV-2 and TDV-3; and 117±6 hours for TDV-4. For tetravalent test articles, plates are incubated at 36±2° C., 5% CO₂ for 93±6 hours for TDV-2 and TDV-3; and 117±6 hours for TDV-1 and TDV-4.

The overlay medium is then decanted from the wells and the cell monolayer is gently washed. The cells are fixed by addition of 1 mL/well of cold 100% methanol and incubated at −20° C. for 20-60 minutes. The fixative is removed and plates are immunostained. Plates may be optionally dried and stored for up to 3 days at room temperature or stored for up to 1 week at 2-8° C. prior to immunostaining. The plates are washed with PBS-T (PBS (phosphate buffer saline) with 0.05% Tween 20) to remove residual fixative. Monoclonal serotype-specific primary antibodies are prepared in Blocking Buffer (PBS-T with 2.5% skim milk and 0.5% Triton X-100), added at 1 mL/well, before incubation at 36±2° C. for 60-90 minutes or overnight at 2-8° C. The primary antibody solution is removed and the plates are washed. A commercial alkaline phosphatase-conjugated goat anti-mouse IgG antibody is diluted and added at 1 mL/well; the plates are incubated at 36±2° C. for 45-75 minutes. The secondary antibody solution is removed, and wells are washed. The dilution of primary and secondary antibodies is determined based on the antibodies qualification. BCIP (5-Bromo-4-chloro-3-indolyl phosphate)/NBT (4-nitro blue tetrazolium chloride) phosphatase substrate, ready-to-use solution is added at 0.5 mL/well and the plates are incubated at room temperature until dark blue or purple foci are clearly visible. The reaction is stopped by washing with distilled water and the plates are allowed to air dry at room temperature. Foci are counted and the virus titer is calculated as Immunofocus plaque forming units per milliliter (PFU/mL) based on the well(s) containing discrete foci that can be accurately counted (10 to 100 foci/well is considered a countable range). Since the foci are detected using serotype-specific monoclonal antibodies, the potency can be determined for each specific serotype of dengue virus in the tetravalent vaccine drug product (DP) samples. The average of the replicates per serotype per drug substance (DS) or in-process sample are reported as log10 PFU/mL. Final DP is in a dosage of 0.5 mL, and the DP potency is reported as log10 PFU/dose.

The viral titers are calculated as follows:
For Takeda's TDV Monovalent Sample (PFU/mL):

$$Titer = \frac{{\sum }_{Wells with 10 to 100 faci} ( Focus count \times Dilution factor )}{\begin{matrix}( Inoculum volume 0.5 mL / well ) \times \\ ( Number of wells with 10 - 100 faci )\end{matrix}}$$

For Takeda's TDV Tetravalent sample (PFU/0.5 ml dose):

$$Titer = \frac{{\sum }_{Wells with 10 to 100 faci} ( Focus count \times Dilution factor )}{\begin{matrix}( Inoculum volume 0.5 mL / well ) \times \\ ( Number of wells with 10 - 100 faci )\end{matrix}} \times 0.5 mL dose$$

### Optimizing Overlay Medium and Concentration—Step 1

It was found that for an overlay medium comprising 0.7% (w/v) high viscosity carboxymethyl cellulose (CMC) (Sigma Aldrich C5013) in water that a large number of blurry and comet-shaped foci resulted. This may have been due to uncontrolled environmental inter-site variations, or manufacturer's batch-to-batch variation of the CMC.

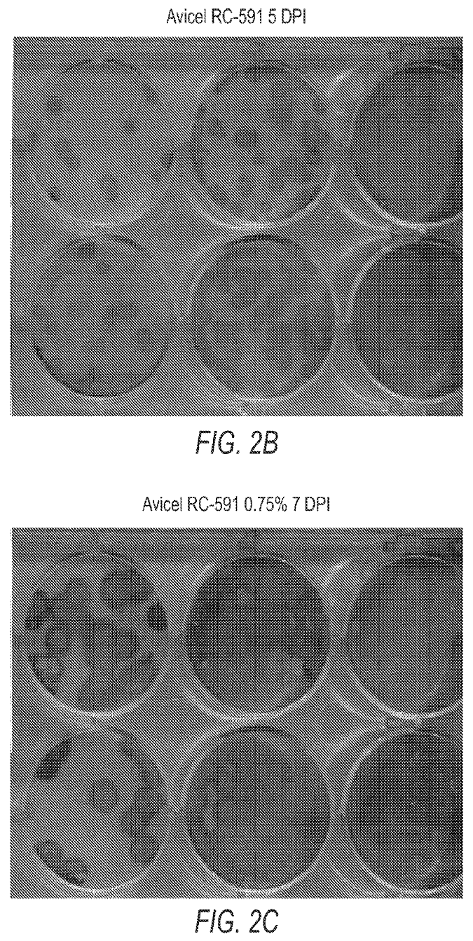

Avicel® RC-591 (proprietary blend of medium viscosity CMC+insoluble microcrystalline cellulose; FMC Corp., DuPont) was evaluated at 0.75% (w/v). Avicel RC/LC are characterized by a content of about 82-89% MCC and about 11 to 18% CMC on a dry basis. An overlay concentration of 0.75% (w/v) Avicel® RC-591 resulted in distinct circular foci, but were too large at 7 days post-infection (dpi) resulting in overlapping foci that could lead to under-estimation of the virus titer. Fixation at 5 dpi resulted in distinct circular foci=2-3 mm in diameter. TDV-1 is shown as a representative example in FIG. 2.

Fixation at 3 and 4 dpi was also tried, and it was determined that at 3 dpi foci are not clearly visible and developed. Day 4 post infection did produce foci that were clearly defined and could be counted without ambiguity.

Later observations resulted in increasing the final Avicel concentration to 1% Avicel RC-591 also provides an ergonomic advantage over CMC as suspensions are not as viscous, allowing removal by decanting (pouring) instead of aspirating one well at a time. It also avoids the problem of residual CMC left behind in the wells during washing, which is extremely sticky and interferes with the immunostaining step.

### Optimization Design—Step 2

Based on the Screening data the factors considered critical were taken into an optimization experiment. The different overlay media (CMC vs. Avicel) had been shown to have similar levels of performance. However, due to the practical benefits of using Avicel as outlined above, Avicel was selected and fixed at 1%.

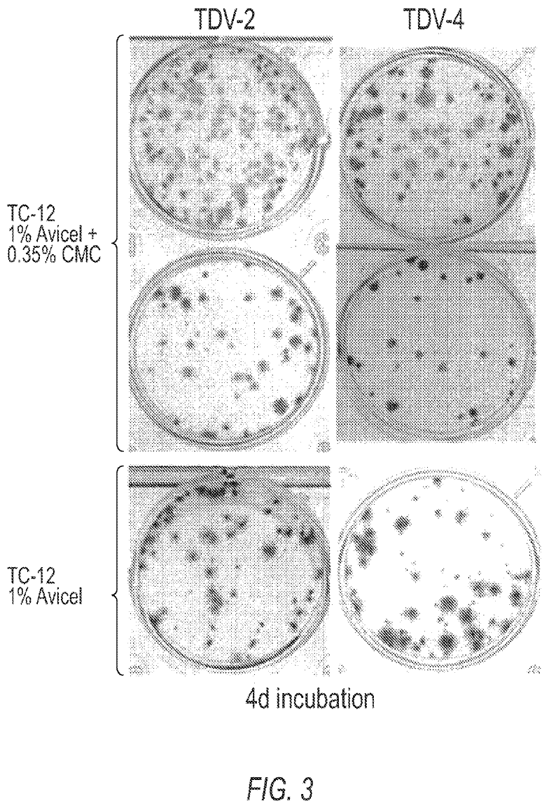

There were some observations that the foci were quite diffuse with many satellites due to insufficient thickness of Avicel alone. Supplementing Avicel with CMC, in particular sodium salt low viscosity CMC, at 0.35% (w/v) can thicken the overlay but also prevent flocculation. FIG. 3 showed that TDV-2 and -4 foci presentation was significantly improved; foci showed less satellites, are sharper and easier to read with 1% (w/v) Avicel+0.35% (w/v) CMC as overlay. Although these experiments were generated in a 12-well plate format, equivalent results are generated in a 6-well format.

### Optimization Design—Step 3: Addition of Non-Ionic Detergent

TDV-4 was titrated in sextuplet on Vero WCB cells. Poloxamer 407 (Pluronic® F 127) was added to the adsorption medium at a final concentration of 0.1% v/v and the adsorption was carried out for 90 min and/or Poloxamer 407 was added to the overlay medium at a final concentration of 0.1% (v/v).

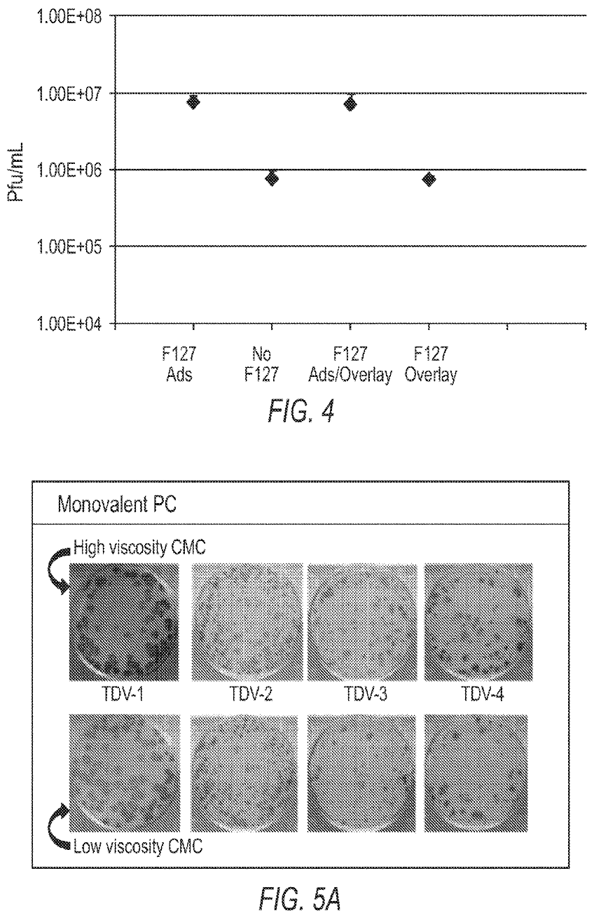

The results are shown in Table 3 and FIG. 4.

TABLE 3

| | F127 Ads | no F127 | F127 ads/OL | F127 OL |
|---|---|---|---|---|
| | 9.00E+06 | 6.00E+05 | 6.00E+06 | 8.00E+05 |
| | 7.00E+06 | 9.00E+05 | 7.00E+06 | 6.00E+05 |
| | 1.00E+07 | 8.00E+05 | 7.00E+06 | 8.00E+05 |
| | 6.00E+06 | 1.00E+06 | 9.00E+06 | 9.00E+05 |
| | 6.00E+06 | 4.00E+05 | 3.00E+06 | 7.00E+05 |
| | 8.00E+06 | 9.00E+05 | 1.10E+07 | 7.00E+05 |
| Average | 7.67E+06 | 7.67E+05 | 7.17E+06 | 7.50E+05 |
| Std | 1632993 | 225092.6 | 2714160.398 | 104880.9 |

Ads: adsorption medium;
OL: overlay medium;
F127: Pluronic ® F127 (Poloxamer 407)

It was observed that by the addition of Poloxamer 407 to the adsorption medium the viral titer was increased by about 10 fold. If Poloxamer 407 was added to the adsorption medium and to the overlay medium, this resulted in an increase of about 9.3 fold compared to the virus titer in the absence of Poloxamer 407. It was further observed that the foci were bigger in the presence of Poloxamer 407 compared to the foci obtained in the absence of Poloxamer 407.

### Comparison of High and Low Viscosity Carboxymethylcellulose in the Overlay Medium for the Immunofocus Assay

A comparison of the immunofocus phenotype using high and low viscosity CMC in the overlay medium was performed. The overlay medium consists of DMEM with 1% Avicel® and 0.35% CMC. In one case high viscosity CMC (Sigma-Aldrich; Cat.-No. C5013) was used, while in the comparison low viscosity CMC (Sigma-Aldrich; Cat.-No. C5678) was used.

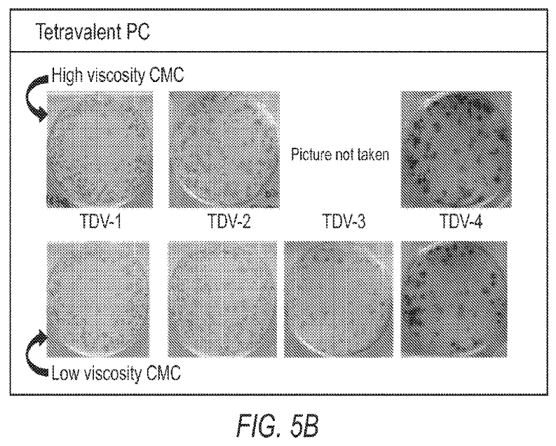

FIGS. 5A and 5B show the foci of each serotype in monovalent and tetravalent positive controls. Visual assessment shows that no difference can be seen between the high and low CMC overlay media preparation in combination with Avicel®.

### Alternative Immunofocus Assay Protocol

Vero cells with a seeding density of 8.31×10⁴ cells/cm² at 36±2° C., 5% CO₂ are grown in 6-well tissue culture for 24 hours to achieve a confluent cell layer (confluence ≥95%). Five-fold serial dilutions of the samples in medium consisting of Dulbecco's Modified Eagle Medium (DMEM) with 1% Pluronic F-127, 1×GlutaMax, 25 mM HEPES, 2% Fetal Bovine Serum (FBS) are prepared, such that the serial dilution brackets the specification range for each virus in the monovalent DS and/or tetravalent DP. For monovalent samples, there are additional pre-dilution steps before the 5-fold serial dilution. Growth medium is removed from the tissue culture plates and 500 μL per well of diluted sample is applied, i.e. one dilution series is applied to each well of the plate. Following virus adsorption for 6±0.5 hours at 36±2° C., 5% CO₂, overlay medium containing DMEM with 2% FBS, 1% (w/v) Avicel® RC-591 and 0.35% (w/v) carboxymethyl cellulose (CMC) sodium salt, medium viscosity (Sigma-Aldrich, Cat. No. C4888), is applied in a volume of 3 mL/well. For monovalent test articles, plates are incubated at 36±2° C., 5% CO₂ for 96±6 hours for TDV-1 to TDV-4. For tetravalent test articles, plates are incubated at 36±2° C., 5% CO₂ for 96±6 hours for TDV-1 to TDV-4.

The overlay medium is then decanted from the wells and the cell monolayer is gently washed. The cells are fixed by addition of 1 mL/well of cold 100% methanol and incubated at <−20° C. for 20-60 minutes. The fixative is removed and plates are immunostained. Plates may be optionally dried and stored for up to 3 days at room temperature or stored for up to 1 week at 2-8° C. prior to immunostaining. The plates are washed with PBS-T (PBS (phosphate buffer saline) with 0.05% Tween 20) to remove residual fixative.

Monoclonal dengue serotype-specific primary antibodies conjugated to alkaline phosphatase (AP) are prepared in Blocking Buffer (PBS-T with 2.5% skim milk and 0.5% Triton X-100), added at 1 mL/well, before incubation at 36±2° C. for 45-75 minutes or overnight at 2-8° C. The primary antibody solution is removed and the plates are washed.

The dilution of primary antibodies is determined based on the antibodies qualification. BCIP (5-Bromo-4-chloro-3-indolyl phosphate)/NBT (4-nitro blue tetrazolium chloride) phosphatase substrate, ready-to-use solution is added at 0.5 mL/well and the plates are incubated at room temperature until dark blue or purple foci are clearly visible. The reaction is stopped by washing with distilled water and the plates are allowed to air dry at room temperature.

1. A method for determining the titer of a dengue virus serotype in a dengue virus-containing sample, the method comprising the steps of:
(a) seeding cells from a dengue-susceptible cell line in an assay plate and culturing the cells for a culture period;
(b) preparing serial dilutions of the dengue virus-containing sample;
(c) adding the serially diluted samples to the cells seeded and cultured in step (a) and incubating the cells over a first incubation period;
(d) providing an overlay medium for the cells incubated in step (c), wherein the overlay medium comprises (i) microcrystalline cellulose and (ii) carboxymethyl cellulose or a salt thereof, and incubating the cells with the overlay medium over a second incubation period;
(e) fixing of the incubated cells;
(f1) immunostaining of the incubated cells using a dengue virus serotype specific antibody as first antibody and a second antibody being specific for the first antibody and conjugated to an enzyme capable of converting a substrate to a visible dye or conjugated to a detectable label; or
(f2) immunostaining of the incubated cells using a dengue virus serotype specific antibody as first antibody conjugated to a detectable label; and
(g) determining the titer of each dengue virus serotype by counting the number of foci in each well of the assay plate.

2. The method according to claim 1, wherein the dengue virus-containing sample comprises at least two different dengue virus serotypes selected from dengue serotype 1, 2, 3 and 4, preferably at least three different serotypes, most preferred the sample comprises four different serotypes.

3. The method according to claim 1 or 2, wherein the overlay medium comprises
(i) microcrystalline cellulose,
(ii) medium viscosity carboxymethyl cellulose or a salt thereof; and
(iii) low or high viscosity carboxymethyl cellulose or a salt thereof, preferably sodium low viscosity carboxymethyl cellulose.

4. The method according to any one of claims 1 to 3, wherein the overlay medium comprises
(a) 0.4% to 1.8% (w/v) microcrystalline cellulose; and
(b) 0.2% to 1.2% (w/v) carboxymethyl cellulose or a salt thereof.

5. The method according to any one of claims 1 to 4, wherein the overlay medium comprises
(a) 0.4% to 1.8% (w/v) microcrystalline cellulose;
(b) 0.05% to 0.7% (w/v) medium viscosity carboxymethyl cellulose or a salt thereof; and
(c) 0.2% to 0.5% (w/v) low viscosity carboxymethyl cellulose or a salt thereof.

6. The method according to any one of claims 1 to 5, wherein the overlay medium in step (d) is prepared by mixing of equal volumes of a 2×Dulbecco's Modified Eagle's Medium (DMEM) solution containing 4% fetal bovine serum (FBS) with a mixture consisting of (a) 2% (w/v) of a suspending agent consisting of about 89% (w/w) microcrystalline cellulose and about 11% (w/w) sodium salt medium viscosity carboxymethyl cellulose and (b) 0.7% (w/v) sodium salt low viscosity carboxymethyl cellulose.

7. The method according to any one of claims 1 to 6, wherein the method is carried out in 6 well or 12 well plates, preferably 6 well plates are used.

8. The method according to any one of claims 1 to 7, wherein the first incubation period in step (c) is 4 to 8 hours.

9. The method according to any one of claims 1 to 8, wherein the first incubation in step (c) is carried out in the presence of a non-ionic detergent, preferably a block copolymer of ethylene oxide and propylene oxide, more preferably Poloxamer 407, and optionally wherein the overlay medium in step (d) additionally contains a non-ionic detergent, preferably a block copolymer of ethylene oxide and propylene oxide, more preferably Poloxamer 407.

10. The method according to claim 9, wherein the final concentration of the non-ionic detergent in the incubation medium is in the range from 0.01% (v/v) to 2% (v/v), preferably from 0.05% (v/v) to 0.15% (v/v), most preferably 0.1% (v/v).

11. The method according to claim 9 or 10, wherein the final concentration of the non-ionic detergent in the overlay medium is in the range from 0.01% (v/v) to 2% (v/v), preferably from 0.05% (v/v) to 0.15% (v/v), most preferably 0.1% (v/v).

12. The method according to any one of claims 1 to 11, wherein in step (d) different second incubation periods are used for the different dengue serotypes.

13. The method according to claim 12, wherein the second incubation period for dengue serotypes 2 and 3 is from 20 to 130 hours, preferably 96±6 hours.

14. The method according to claim 12, wherein the second incubation period for dengue serotypes 1 and 4 is from 20 to 130 hours, preferably 120±6 hours.

15. The method according to any one of claims 1 to 14, wherein the dengue-susceptible cell line is selected from Vero cells, LLC-MK2 cells and BHK-21 cells, preferably the dengue-susceptible cell line is a Vero cell line derived from ATCC CCL-81.

16. The method according to any one of claims 1 to 15, wherein the culture period in step (a) is such that the cell monolayers are at least 90% confluent.

17. The method according to any one of claims 2 to 16, wherein the dengue serotype 1 is DENV-1 strain 16007, dengue serotype 2 is DENV-2 strain 16681, dengue serotype 3 is DENV-3 strain 16562 and dengue serotype 4 is DENV-4 strain 1036.

18. The method according to any one of claims 1 to 17, wherein in step (c) the cells are incubated at a temperature of 34° C. to 38° C.

19. The method according to any one of claims 1 to 18, wherein said sample is a vaccine comprising a monovalent dengue virus composition.

20. The method according to any one of claims 1 to 18, wherein said sample is a vaccine comprising a multivalent dengue virus composition, preferably a tetravalent dengue virus composition.

21. The method according to claim 19 or 20, wherein the dengue virus vaccine comprises a chimeric dengue serotype 2/1 strain, a dengue serotype 2 strain, a chimeric dengue serotype 2/3 strain, and a chimeric dengue serotype 2/4 strain.

22. The method according to any one of claims 1 to 18, wherein said sample is a sample from an individual potentially infected with dengue virus.

23. Use of the method according to any one of claims 1 to 22 in the quality control of a virus preparation or a vaccine composition.

24. Use of the method according to claim 23, wherein the vaccine composition is TAK-003 (QDENGA®).

25. Use of the method according to any one of claims 1 to 22 in the diagnosis of a blood sample from a Dengue infected individual.


SEQUENCE LISTING (4 sequences)

SEQ ID NO: 1 - protein, 3391 aa, synthetic construct
  source 1..3391: note = chimeric dengue serotype 1 (MVS)
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRGGEPHMIVSKQERGKSLLFKTSAGVNMCTLIAMDLGELCEDTMTYKCPRITEAEPDDVDCWCNATDTWVTYGTCSQTGEHRRDKRSVALAPHVGLGLETRAETWMSSEGAWKQIQKVETWALRHPGFTVIALFLAHAIGTSITQKGIIFILLMLVTPSMAMRCVGIGNRDFVEGLSGATWVDVVLEHGSCVTTMAKNKPTLDIELLKTEVTNPAVLRKLCIEAKISNTTTDSRCPTQGEATLVEEQDANFVCRRTFVDRGWGNGCGLFGKGSLITCAKFKCVTKLEGKIVQYENLKYSVIVTVHTGDQHQVGNETTEHGTTATITPQAPTSEIQLTDYGTLTLDCSPRTGLDFNEMVLLTMKERSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSTQDEKGATQNGRLITANPIVTDKEKPVNIEAEPPFGESYIVVGAGEKALKLSWFKKGSSIGKMFEATARGARRMAILGDTAWDFGSIGGVFTSMGKLVHQVFGTAYGVLFSGVSWTMKIGIGILLTWLGLNSRNTSLSMMCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTLPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEDQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 2 - protein, 3391 aa, Dengue virus
  source 1..3391: note = Dengue virus serotype 2 (MVS)
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRNGEPHMIVSRQEKGKSLLFKTEVGVNMCTLMAMDLGELCEDTITYECPLLRQNEPEDIDCWCNSTSTWVTYGTCTTMGEHRREKRSVALVPHVGMGLETRTETWMSSEGAWKHVQRIETWILRHPGFTMMAAILAYTIGTTHFQRALIFILLTAVTPSMTMRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMFETTMRGAKRMAILGDTAWDFGSLGGVFTSIGKALHQVFGAIYGAAFSGVSWTMKILIGVIITWIGMNSRSTSLSVTLVLVGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAVFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 3 - protein, 3389 aa, synthetic construct
  source 1..3389: note = chimeric Dengue virus serotype 3 (MVS)
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRRSAGMIIMLIPTVMAFHLTTRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMTMRCVGVGNRDFVEGLSGATWVDVVLEHGGCVTTMAKNKPTLDIELQKTEATQLATLRKLCIEGKITNITTDSRCPTQGEAILPEEQDQNYVCKHTYVDRGWGNGCGLFGKGSLVTCAKFQCLESIEGKVVQHENLKYTVIITVHTGDQHQVGNETQGVTAEITPQASTAEAILPEYGTLGLECSPRTGLDFNEMISLTMKNKAWMVHRQWFFDLPLPWTSGASAETPTWNRKELLVTFKNAHAKKQEVVVLGSQEGAMHTALTGATEIQTSGGTSIFAGHLKCRLKMDKLELKGMSYAMCLSSFVLKKEVSETQHGTILIKVEYKGEDAPCKIPFSTEDGQGKALNGRLITANPVVTKKEEPVNIEAEPPFGESNIVIGIGDKALKINWYKKGSSIGKMFEATARGARRMAILGDTAWDFGSVGGVLNSLGKMVHQIFGSAYTALFGGVSWMMKIGIGVLLTWIGLNSKNTSMSFSCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTDDIGMGVTYLALLAAFKVRPTFAAGLLLRKLTSKELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKARDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW

SEQ ID NO: 4 - protein, 3391 aa, synthetic construct
  VARIANT 1226: note = X is K or R
  source 1..3391: note = chimeric Dengue virus serotype 4 (MVS)
MNNQRKKAKNTPFNMLKRERNRVSTVQQLTKRFSLGMLQGRGPLKLFMALVAFLRFLTIPPTAGILKRWGTIKKSKAINVLRGFRKEIGRMLNILNRRRSSAGMIIMLIPTVMAFHLTTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYGMRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRIISSTPLAENTNSVTNIELEPPFGDSYIVIGVGNSALTLHWFRKGSSIGKMFESTYRGAKRMAILGETAWDFGSVGGLFTSLGKAVHQVFGSVYTTLFGGVSWMIRILIGFLVLWIGTNSRNTSMAMTCIAAGIVTLYLGVMVQADSGCVVSWKNKELKCGSGIFITDNVHTWTEQYKFQPESPSKLASAIQKAHEEDICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAGHGQVDNFSLGVLGMALFLEEMLRTRVGTKHAILLVAVSFVTLITGNMSFRDLGRVMVMVGATMTGDIGMGVTYLALLAAFKVRPTFAAGLLLRKLTSXELMMTTIGIVLLSQSTIPETILELTDALALGMMVLKMVRNMEKYQLAVTIMAILCVPNAVILQNAWKVSCTILAVVSVSPLFLTSSQQKTDWIPLALTIKGLNPTAIFLTTLSRTSKKRSWPLNEAIMAVGMVSILASSLLKNDIPMTGPLVAGGLLTVCYVLTGRSADLELERAADVKWEDQAEISGSSPILSITISEDGSMSIKNEEEEQTLTILIRTGLLVISGLFPVSIPITAAAWYLWEVKKQRAGVLWDVPSPPPMGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRRLTIMDLHPGAGKTKRYLPAIVREAIKRGLRTLILAPTRVVAAEMEEALRGLPIRYQTPAIRAVHTGREIVDLMCHATFTMRLLSPVRVPNYNLIIMDEAHFTDPASIAARGYISTRVEMGEAAGIFMTATPPGSRDPFPQSNAPIIDEEREIPERSWNSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGVKNNQILEENVEVEIWTKEGERKKLKPRWLDARIYSDPLALKEFKEFAAGRKSLTLNLITEMGRLPTFMTQKVRDALDNLAVLHTAEAGGRAYNHALSELPETLETLLLLTLLATVTGGIFLFLMSARGIGKMTLGMCCIITASILLWYAQIQPHWIAASIILEFFLIVLLIPEPEKQRTPQDNQLTYVVIAILTVVAATMANEMGFLEKTKKDLGLGSIATQQPESNILDIDLRPASAWTLYAVATTFVTPMLRHSIENSSVNVSLTAIANQATVLMGLGKGWPLSKMDIGVPLLAIGCYSQVNPITLTAALFLLVAHYAIIGPGLQAKATREAQKRAAAGIMKNPTVDGITVIDLDPIPYDPKFEKQLGQVMLLVLCVTQVLMMRTTWALCEALTLATGPISTLWEGNPGRFWNTTIAVSMANIFRGSYLAGAGLLFSIMKNTTNTRRGTGNIGETLGEKWKSRLNALGKSEFQIYKKSGIQEVDRTLAKEGIKRGETDHHAVSRGSAKLRWFVERNMVTPEGKVVDLGCGRGGWSYYCGGLKNVREVKGLTKGGPGHEEPIPMSTYGWNLVRLQSGVDVFFIPPEKCDTLLCDIGESSPNPTVEAGRTLRVLNLVENWLNNNTQFCIKVLNPYMPSVIEKMEALQRKYGGALVRNPLSRNSTHEMYWVSNASGNIVSSVNMISRMLINRFTMRYKKATYEPDVDLGSGTRNIGIESEIPNLDIIGKRIEKIKQEHETSWHYDQDHPYKTWAYHGSYETKQTGSASSMVNGVVRLLTKPWDVVPMVTQMAMTDTTPFGQQRVFKEKVDTRTQEPKEGTKKLMKITAEWLWKELGKKKTPRMCTREEFTRKVRSNAALGAIFTDENKWKSAREAVEDSRFWELVDKERNLHLEGKCETCVYNMMGKREKKLGEFGKAKGSRAIWYMWLGARFLEFEALGFLNEDHWFSRENSLSGVEGEGLHKLGYILRDVSKKEGGAMYADDTAGWDTRITLEDLKNEEMVTNHMEGEHKKLAEAIFKLTYQNKVVRVQRPTPRGTVMDIISRRDQRGSGQVGTYGLNTFTNMEAQLIRQMEGEGVFKSIQHLTITEEIAVQNWLARVGRERLSRMAISGDDCVVKPLDDRFASALTALNDMGKIRKDIQQWEPSRGWNDWTQVPFCSHHFHELIMKDGRVLVVPCRNQDELIGRARISQGAGWSLRETACLGKSYAQMWSLMYFHRRDLRLAANAICSAVPSHWVPTSRTTWSIHAKHEWMTTEDMLTVWNRVWIQENPWMEDKTPVESWEEIPYLGKREDQWCGSLIGLTSRATWAKNIQAAINQVRSLIGNEEYTDYMPSMKRFRREEEEAGVLW